(19) United States
(12) Patent Application Publication
(10) Pub. No.: US 2020/0085524 A1
(43) Pub. Date: Mar. 19, 2020

(54) SURGICAL SLEEVE FOR ROBOTIC SYSTEMS

(71) Applicant: Covidien LP, Mansfield, MA (US)

(72) Inventors: Arvind Ramadorai, Lexington, MA (US); Joshua Snow, Clinton, CT (US); Jacqueline Jones, Hamden, CT (US)

(21) Appl. No.: 16/615,745

(22) PCT Filed: May 8, 2018
(86) PCT No.: PCT/US2018/031537

§ 371 Date: Nov. 21, 2019

Related U.S. Application Data
(60) Provisional application No. 62/510,544, filed on May 24, 2017.

(51) Int. Cl. A61B 46/10; A61B 50/30; A61B 90/00

(52) U.S. Cl. CPC A61B 46/10; A61B 34/30; A61B 90/08; A61B 50/30

(57) ABSTRACT

A surgical sleeve includes a sheath and a contact surface. The sheath defines a lumen therein. The lumen is configured to receive at least a portion of a surgical instrument therein. The sheath is expandable to transition from an un-deployed configuration to a deployed configuration. As the sheath transitions from the un-deployed configuration to the deployed configuration, a length of the lumen increases. The contact surface is disposed along at least a portion of the sheath and is configured to dampen an impact force thereon.

### BACKGROUND

Robotic surgical systems or “Telesurgery” used in minimally invasive medical procedures may include a console or cart supporting a robot arm and a surgical instrument, for example, a trocar, a forceps, a stapler, or a grasping tool. The robotic arm provides mechanical and/or electrical power to the surgical instrument for its operation and movement. During a medical procedure, the surgeon typically operates a controller which remotely controls the articulation and actuation of the robotic arm and/or the surgical instrument at the surgical site from a location that may differ from the patient.

During articulation and/or actuation of the robotic arm and/or surgical instrument, incidental contact may occur between the robotic arm or surgical instrument and the patient or surgical staff present within the operating theater. As a result of such contacts, unintended consequences may occur, such as, for example, damage to the robotic arm, surgical instrument, or accompanying assemblies; a degrading or rendering of sterile drapes or patient coverings inoperable; and/or the need to re-sterilize the surgical site, the robotic surgical system, or the surgical instrument during the pendency of a procedure.

Accordingly, new surgical sleeves and coverings which may be easily and efficiently installed and removed are desired which inhibit, absorb, or dampen an impact force derived from incidental physical contact between the patient or surgical staff and the robotic surgical systems or components thereof.

### SUMMARY

A surgical sleeve in accordance with the present disclosure is used to enshroud surgical tools or components, which may be coupled to a robotic surgical system, such that any incidental impact forces asserted thereon are absorbed or dampened.

In accordance with an aspect of the present disclosure, a surgical sleeve includes a sheath and a contact surface. The sheath defines a lumen therein. The lumen is configured to receive at least a portion of a surgical instrument therein. The sheath is expandable to transition from an un-deployed configuration to a deployed configuration. A length of the lumen increases as the sheath transitions from the un-deployed configuration to the deployed configuration. The contact surface is disposed along at least a portion of the sheath, and is configured to dampen an impact force thereon.

In an embodiment, the sheath is furled onto itself in the un-deployed configuration, and unfurls as the sheath transitions towards the deployed configuration. In another embodiment, the contact surface includes a resilient foam element. In an embodiment, the contact surface includes a rubber element. In yet another embodiment, the sheath includes a material configured to withstand sterilization. In yet another embodiment, the sheath includes a material configured to maintain the lumen of the sheath in a sterile condition. In an embodiment, the sheath defines at least one port therethrough. The port is configured to receive a portion of the surgical instrument therethrough.

In another embodiment, the sheath includes a contact sensor thereon. The contact sensor is configured to measure a magnitude of force or detect the presence of physical contact upon the sheath. In a further embodiment, the contact sensor is supported on the contact surface.

In accordance with another aspect of the present disclosure, a surgical wrap is provided and includes a sheath and a contact surface. The sheath includes a longitudinal seam and defines a first radial end portion and a second radial end portion. The sheath is transitionable between an un-deployed configuration and a deployed configuration. In the deployed configuration, the first radial end portion of the sheath affixes to the second radial end portion of the sheath such that in the deployed configuration the sheath defines a lumen therethrough configured to enshroud at least a portion of a surgical instrument. The contact surface is disposed along at least a portion of the sheath and is configured to dampen an impact force thereon.

In an embodiment, the contact surface includes a resilient foam element. In another embodiment, the contact surface includes a rubber element. In yet another embodiment, the sheath includes a material configured to withstand sterilization.

In another embodiment, the sheath includes a contact sensor thereon. The contact sensor is configured to measure a magnitude of force or detect physical contact upon the sheath. In a further embodiment, the contact sensor is supported on the contact surface.

In another embodiment, the sheath or contact surface includes a contact sensor thereon. The contact sensor can be used to maintain a specified magnitude of force

In another embodiment, at least one of the first or second radial end portions of the sheath further includes an affixation element. The affixation element is configured to selectively affix the first radial end portion to the second radial end portion in the deployed configuration. In a further embodiment, the affixation element includes a string. In an embodiment, the affixation element includes an adhesive.

In a further embodiment, the lumen of the sheath defines a shape complementary to the surgical instrument. In yet another embodiment, the sheath includes a material configured to maintain the lumen of the sheath in a sterile condition.

In another embodiment, the sheath defines at least one port therethrough. The port is configured to receive a portion of a surgical instrument therethrough.

In accordance with yet another aspect of the present disclosure, a kit includes at least one surgical sleeve and an instruction for use of the at least one surgical sleeve. The surgical sleeve includes a sheath and a contact surface. The sheath defines a lumen therein. The lumen is configured to receive at least a portion of a surgical instrument therein. The sheath is expandable to transition from an un-deployed configuration to a deployed configuration, such that a length of the lumen increases as the sheath transitions from the un-deployed configuration to the deployed configuration. The contact surface is disposed along at least a portion of the sheath, and is configured to dampen an impact force thereon.

The kit further includes an instruction for directing a user to position the at least one surgical sleeve over a surgical instrument or transition the at least one surgical sleeve from the un-deployed configuration to the deployed configuration.

In an embodiment, the kit further includes at least one surgical instrument including a trocar or a trocar sleeve.

In another embodiment, the kit further includes a sterile packaging configured to retain the at least one surgical sleeve and the instruction for use.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a schematic illustration of a robotic surgical system in accordance with the present disclosure;

FIG. 2 is a perspective view of a robotic arm having a trocar mount coupled thereto, with a trocar uncoupled from the trocar mount;

FIG. 3A is a side, perspective view of a surgical sleeve in accordance with the present disclosure, shown in an un-deployed configuration;

FIG. 3B is a side, perspective view of the surgical sleeve of FIG. 3A, shown in a deployed configuration;

FIG. 3C is a side, perspective view of the surgical sleeve of FIG. 3B, shown shrouding the trocar of FIG. 2;

FIG. 4A is a side, perspective view of another embodiment of a surgical sleeve in accordance with the present disclosure, shown in an un-deployed configuration;

FIG. 4B is a side, perspective view of the surgical sleeve of FIG. 4A, shown in a deployed configuration; and

FIG. 5 is a schematic illustration of a kit in accordance with the present disclosure.

### DETAILED DESCRIPTION

Embodiments of the present disclosure are described in detail with reference to the drawings, in which like reference numerals designate identical or corresponding elements in each of the several views. As used herein, the term “distal” refers to that portion of a device that is farther from the user.

While robotic surgical systems, or “Telesurgery”, are discussed below, the embodiments disclosed herein may be configured to work with traditional instruments used during open surgery, minimally invasive instruments, or with any instrument or tool where incidental physical contact may occur. Only for brevity the features of the device disclosed herein will be directed towards robotic surgical systems. Robotic surgical systems employ various robotic elements to assist the surgeon and allow remote operation (or partial remote operation) of surgical instrumentation. Various robotic arms, gears, cams, pulleys, electric and mechanical motors, etc. may be employed for this purpose and may be designed with a robotic surgical system to assist the surgeon during the course of an operation or treatment. Such robotic systems may include remotely steerable systems, automatically flexible surgical systems, remotely flexible surgical systems, remotely articulating surgical systems, wireless surgical systems, modular or selectively configurable remotely operated surgical systems, etc.

The robotic surgical systems may be employed with one or more consoles that are next to the operating theater or located in a remote location. In this instance, one team of surgeons or nurses may prep the patient for surgery and configure the robotic surgical system with one or more of the instruments disclosed herein while another surgeon (or group of surgeons) remotely controls the instruments via the robotic surgical system. As can be appreciated, a highly skilled surgeon may perform multiple operations in multiple locations without leaving his/her remote console which can be both economically advantageous and a benefit to the patient or a series of patients.

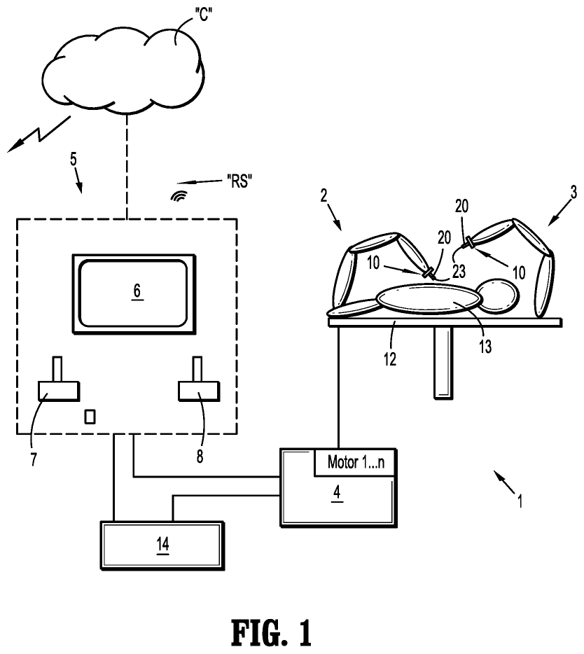

With reference to FIG. 1, a robotic surgical system 1 includes a plurality of robotic arms 2, 3; a control device 4; and an operating console 5 coupled with control device 4. Operating console 5 includes a display device 6, which may be set up in particular to display three-dimensional images; and manual input devices 7, 8, by means of which a person (not shown), for example a surgeon, is able to telemanipulate robotic arms 2, 3.

Each of the plurality of robotic arms 2, 3 includes a plurality of members, which are connected through joints. Robotic surgical system 1 also includes a surgical assembly 10 connected to a distal end of each of robotic arms 2, 3. Surgical assembly 10 includes an instrument drive unit and a surgical instrument 20 detachably coupled to instrument drive unit. Surgical instrument 20 includes an end effector 23.

Robotic arms 2, 3 may be driven by electric drives (not shown) that are connected to control device 4. Control device 4 (e.g., a computer) is set up to activate the drives, in particular by means of a computer program, in such a way that robotic arms 2, 3, their surgical assemblies 10 execute a desired movement according to a movement defined by means of manual input devices 7, 8. Control device 4 may also be set up in such a way that it regulates movement of robotic arms 2, 3 and/or of the drives.

With continued reference to FIG. 1, robotic surgical system 1 is configured for use on a patient 13 lying on a patient table 12 to be treated in a minimally invasive manner by means of end effector 23. Robotic surgical system 1 may include more than two robotic arms 2, 3. The additional robotic arms may also be connected to control device 4 and may be telemanipulatable by means of operating console 5. One or more additional surgical assemblies 10 and/or surgical instruments 20 may also be attached to the additional robotic arm.

Control device 4 may control a plurality of motors (Motor 1 . . . n) with each motor configured to drive a pushing or a pulling of one or more cables (not shown) coupled to end effector 23 of surgical instrument 20. It is also contemplated that the cables can be replaced with rods or the like. In use, as these cables are pushed and/or pulled, the cables effect operation and/or movement of end effector 23 of surgical instrument 20. It is contemplated that control device 4 coordinates the activation of the various motors (Motor 1 . . . n) to coordinate a pushing or a pulling motion of one or more of the cables in order to coordinate an operation and/or movement of one or more end effectors 23. In embodiments, each motor can be configured to actuate a drive rod or a lever arm to effect operation and/or movement of end effectors 23 in addition to, or instead of, one or more cables.

Control device 4 can include any suitable logic control circuit adapted to perform calculations and/or operate according to a set of instructions. Control device 4 can be configured to communicate with a remote system “RS”, either via a wireless (e.g., Wi-Fi™, Bluetooth®, LTE™, etc.) and/or wired connection. Remote system “RS” can include data, instructions and/or information related to the various components, algorithms, and/or operations of robotic surgical system 1. Remote system “RS” can include any suitable electronic service, database, platform, cloud “C” (see FIG. 1), or the like. Control device 4 may include a central processing unit operably connected to memory. The memory may include transitory type memory (e.g., RAM) and/or non-transitory type memory (e.g., flash media, disk media, etc.). In some embodiments, the memory is part of, and/or operably coupled to, remote system “RS”.

Control device 4 can include a plurality of inputs and outputs for interfacing with the components of robotic surgical system 1, such as through a driver circuit. Control device 4 can be configured to receive input signals and/or generate output signals to control one or more of the various components (e.g., one or more motors) of robotic surgical system 1. The output signals can include, and/or can be based upon, algorithmic instructions which may be pre-programmed and/or input by a user. Control device 4 can be configured to accept a plurality of user inputs from a user interface (e.g., switches, buttons, touch screen, etc. of operating console 5) which may be coupled to remote system “RS”.

A database 14 can be directly and/or indirectly coupled to control device 4. Database 14 can be configured to store pre-operative data from living being(s) and/or anatomical atlas(es). Database 14 can include memory which can be part of, and/or operatively coupled to, remote system “RS”. Reference may be made to U.S. Pat. No. 8,828,023, filed on Nov. 3, 2011, entitled “Medical Workstation,” the entire content of which is incorporated herein by reference, for a detailed discussion of the construction and operation of robotic surgical system 1.

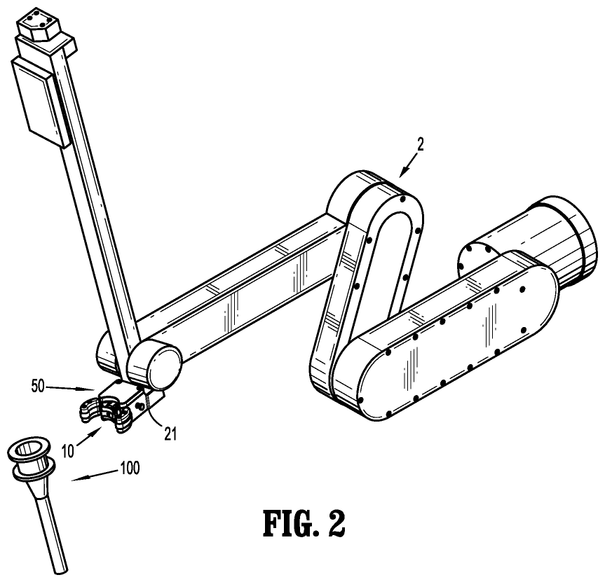

Turning now to FIG. 2, surgical assembly 10 includes an exemplary embodiment of surgical instrument 20 (FIG. 1) illustrated as trocar mount 50, where trocar mount 50 is releasably coupled to robotic arm 2 and serves to releasably couple a trocar or trocar sleeve 100 thereto. It should be appreciated that trocar or trocar sleeve 100 and trocar mount 50 may represent any surgical tool or component utilized during minimally invasive or open surgery which may be coupled to robotic surgical system 1, such as, for example, forceps, staplers, graspers, cutters, biopsy probes, imaging probes, radiation probes, etc., wherein trocar or trocar sleeve 100 and trocar mount 50 will be discussed herein for the sake of brevity.

Trocar mount 50 is configured to releasably couple trocar or trocar sleeve 100 to robotic arm 2, such that additional surgical tools may be introduced through trocar or trocar sleeve 100 during a surgical procedure. During such a surgical procedure, articulation of robotic arms 2, 3, or any other surgical instrument or component complementary to robotic surgical system 1, may cause trocar or trocar sleeve 100, trocar mount 50, and/or instruments passed therethrough, to make inadvertent contact with the patient, the surgical staff, or other components of robotic surgical system 1. Accordingly, robotic surgical system 1 may include a surgical sleeve, drape, or wrap which encloses or enshrouds trocar or trocar sleeve 100, trocar mount 50, robotic arm 2, any other components or assemblies of robotic surgical system 1, and/or any combination thereof, such that impact forces derived from incidental physical contact therebetween is inhibited, absorbed, or dampened.

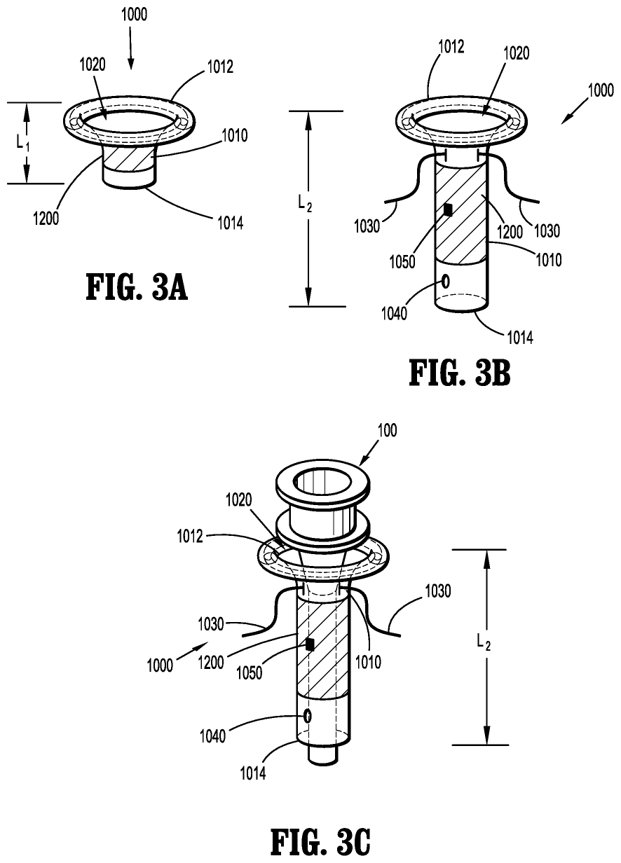

With reference to FIGS. 3A-3C, a surgical sleeve 1000 is shown covering or essentially enclosing or enshrouding at least a portion of trocar or trocar sleeve 100. In use with robotic surgical system 1, surgical sleeve 1000 may be configured to be: positioned over trocar or trocar sleeve 100 prior to or after mounting trocar or trocar sleeve 100 to trocar mount 50; positioned over trocar mount 50 prior to or after mounting trocar mount 50 to robotic arm 2, or prior to or after mounting trocar or trocar sleeve 100 to trocar mount 50; positioned over both trocar or trocar sleeve 100 and trocar mount 50; or positioned over trocar or trocar sleeve 100, trocar mount 50, and robotic arm 2. For the sake of brevity, surgical sleeve 1000 will be described herein with respect to trocar or trocar sleeve 100 and trocar mount 50. During use, surgical sleeve 1000 acts to inhibit, absorb, or dampen impact forces derived from incidental physical contact between trocar or trocar sleeve 100 and surrounding objects, such as, for example, the patient, the surgical staff, or other components of robotic surgical system 1.

Surgical sleeve 1000 includes a sheath 1010 and at least one contact surface 1200 disposed thereon. Sheath 1010 includes a first open end 1012, a second open end 1014, and a cavity or lumen 1020 therebetween. Cavity or lumen 1020 is configured to receive trocar or trocar sleeve 100 therein, or alternatively, any other surgical or robotic tool, component, or assembly as described herein. In accordance with an embodiment of the present disclosure, sheath 1010 may be loose fitting, or may alternatively have a pre-set or pre-defined shape having defined dimensions which are complementary to trocar or trocar sleeve 100, or any surgical tool or component received with cavity or lumen 1020.

Sheath 1010 may be configured to radially expand and/or axially extend such that sheath 1010, and thus surgical sleeve 1000, transitions between an un-deployed configuration (FIG. 3A) and a deployed configuration (3B). In the un-deployed configuration, sheath 1010 is folded, rolled, or otherwise furled about self, such that a length “L₁” of cavity or lumen 1020 is defined. As sheath 1010 transitions from the un-deployed configuration towards the deployed configuration, a portion of sheath 1010 may unfold, unroll, or otherwise unfurl such that the length of cavity or lumen 1020 expands or extends from first length “L₁,” in the un-deployed configuration, towards a second, larger length “L₂,” in the deployed configuration. It should be appreciated that sheath 1010 may similarly transition from the deployed configurations towards the un-deployed configuration, whereby the length of cavity or lumen 1020 is reduces from the second, larger length “L₂” towards the first length “L₁.” Further, second, larger length “L₂” may be user dependent such that the length of cavity or lumen 1020 may be set at any length desired by an operator.

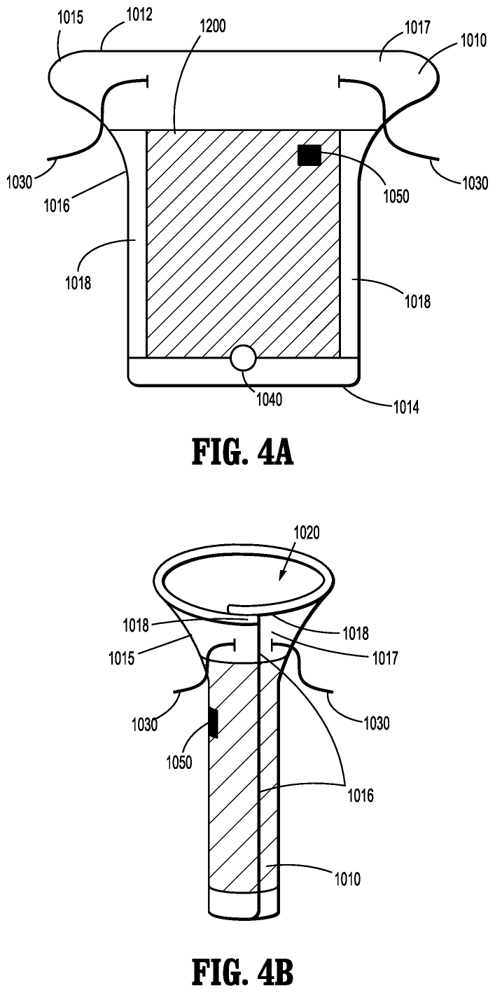

In an embodiment, and with reference momentarily to FIGS. 4A and 4B, alternatively or additionally to unfolding, unrolling, or unfurling sheath 1010 such that the length of cavity or lumen 1020 expands or extends to enclose or enshroud trocar or trocar sleeve 100, sheath 1010 may be configured to wrap around trocar or trocar sleeve 100. In such an embodiment, sheath 1010 may include a longitudinal seam 1016 such that in the un-deployed configuration a first radial end portion 1015 of sheath 1010 may be wrapped around trocar or trocar sleeve 100 and affixed to a second radial end portion 1017 of sheath 1010. With first radial end portion 1015 of sheath 1010 affixed to second radial end portion 1017 of sheath 1010, cavity or lumen 1020 of sheath 1010 is defined, and sheath 1010 assumes the deployed configuration. Accordingly, in such an embodiment, in the un-deployed configuration, where first and second radial end portions 1015, 1017 of sheath 1010 are not affixed, sheath 1010 may define a generally planar configuration. In the deployed configuration, with first and second radial end portions 1015, 1017 affixed to one another, sheath 1010 defines cavity or lumen 1020 therethrough such that trocar or trocar sleeve 100 may be enclosed thereby. At least one of first radial end portion 1015 or second radial end portion 1017 may further include an affixation element 1018 such that first and second radial end portions 1015, 1017 may be selectively affixed to one another. Affixation element 1018 may be disposed along a portion of, or the entirely of longitudinal seam 1017, and may define an adhesive element, a tie string, a tow-part hook and look type fastener, or any other means known in the art such that first radial end portion 1015 may be selectively affixed to second radial end portion 1017.

With reference to FIGS. 3A-4B, the contact surface 1200 of surgical sleeve 1000 is disposed over at least a portion of sheath 1010. It is envisioned that contact surface 1200 may be disposed over the entirety of sheath 1010, or that several contact surfaces may be provided over multiple portions of the sheath, and may assume any pattern or geometric configuration to inhibit, absorb, or dampen an impact force on the sheath of surgical instrument positioned therein. Further, contact surface 1200 may be disposed about sheath 1010 such that contact surface 1200 circumscribes a portion of, or the entirety of lumen 1020 defined by sheath 1010, extends longitudinally along a portion of, or the entirety of second length “L₂” of lumen 1020. As an exemplary illustration, FIGS. 3B-4B show contact surface 1200 circumscribing lumen 1020 of sheath 1010 and extending longitudinally along a portion of sheath 1010 with sheath 1010 in the deployed configuration.

Contact surface 1200 is configured to define a resilient or compressible portion of sheath 1010, and more particularly may include a flexible element, a resilient foam element, a rubber element, a gel element, or any combination thereof, such that contact surface 1200 inhibits, absorbs, or dampens an impact force thereon. It is further envisioned that contact surface 1200 may be configured to act as a crumple zone for surgical sleeve 1000, such that contact surface 1200 is destructively deformed, thus absorbing and dampening an impact force thereon.

In use, surgical sleeve 1000 is brought into approximation with an end of trocar or trocar sleeve 100. With surgical sleeve 1000 in the un-deployed configuration (FIGS. 3A and 4A), trocar or trocar sleeve 100 may be inserted into cavity or lumen 1020 of sheath 1010, after which, surgical sleeve 1000 may be transitioned into the deployed configuration (FIGS. 3B and 4B). Alternatively, surgical sleeve 1000 may be first transitioned into the deployed configuration, whereby trocar or trocar sleeve 100 is next inserted into cavity or lumen 1020 of sheath 1010 with surgical sleeve 1000 in the deployed configuration. In either embodiment, surgical sleeve 1000 may be incrementally transitioned from the un-deployed configuration towards the deployed configuration as required by the operator. Further, as surgical sleeve 1000 is transitioned towards the deployed configuration, the second, larger length “L₂” of cavity or lumen 1020 may be tailored to accommodate for a given length of trocar or trocar sleeve 100, or any alternative surgical object inserted within cavity or lumen 1020. Once surgical sleeve 1000 is deployed, contact surface 1200 of surgical sleeve 1000 acts to inhibit, absorb, and dampen any incidental impact forces which may be imparted upon trocar or trocar sleeve 100.

As discussed briefly above, in use with robotic surgical system 1, surgical sleeve 1000 may be configured to be positioned over: trocar or trocar sleeve 100; trocar mount 50; both trocar or trocar sleeve 100 and trocar mount 50; or trocar or trocar sleeve 100, trocar mount 50, and robotic arm 2. In embodiments, when deployed, cavity or lumen 1020 of sheath 1010 of surgical sleeve 1000 may define a complementary length and/or radial diameter to receive trocar or trocar sleeve 100, trocar mount 50, or robotic arm 2. Alternatively, one or more surgical sleeves 1000 may be utilized to individually enclose or enshroud trocar or trocar sleeve 100, trocar mount 50, or robotic arm 2.

Surgical sleeve 1000 may be formed from any biocompatible material known in the art which provides flexibility to enable ease of transition from the un-deployed configuration towards the deployed configuration thereof, and permits unobstructed movement of the covered surgical instrument, e.g., trocar or trocar sleeve 100, while maintaining a strong tear resistant barrier. For example, surgical sleeve 1000 may be fabricated from a flexible and/or impermeable material, such as, for example, plastics, polymers, polymer blends, thermoplastics, combinations thereof, or the like. Surgical sleeve 1000 may be adapted to form fit or loosely fit over covered surgical instrument, e.g., trocar or trocar sleeve 100, trocar mount 50, robotic arm 2, and/or other components of robotic surgical system 1, or made to freely stretch or bend to their respective movements during use. Further, surgical sleeve 1000 may be configured for sterilization, such that the surgical sleeve 1000 may act as a sterile barrier to prevent contamination of trocar or trocar sleeve 100, trocar mount 50, and/or robotic arm 2 (e.g., from bodily fluids, ambient environment, etc.). In such an embodiment, once deployed, surgical sleeve 1000 may act to provide a sterile barrier between the operating theater and trocar or trocar sleeve 100, trocar mount 50, or robotic arm 2. Further still, surgical sleeve 1000 may be configured to be reusable, or alternatively, to be disposable.

It is further envisioned that sheath 1010 of surgical sleeve 1000 may be biased radially inward, such that sheath 1010 cinches upon, or constrains around trocar or trocar sleeve 100 when disposed within cavity or lumen 1020 thereof, and such that longitudinal translation of surgical sleeve 1000 with respect to trocar or trocar sleeve 100 is inhibited following deployment. Alternatively, or additionally, surgical sleeve 1000 may include an attachment element 1030 disposed upon sheath 1010 and configured to engage trocar or trocar sleeve 100, such that longitudinal translation of surgical sleeve 1000 with respect to trocar or trocar sleeve 100 is inhibited following deployment thereof. Attachment element 1030 may define a tie, a string, an adhesive, an elastic element, or any other attachment element as is known in the art such that surgical sleeve 1000 may be fixed with respect to trocar or trocar sleeve 100. It is further envisioned that attachment element 1030 may act to inhibit surgical sleeve 1000 from inadvertently transitioning from the deployed configuration towards the un-deployed configuration prior to the completion of the procedure.

Further still, it is contemplated that surgical sleeve 1000 may define at least one port 1040 extending through sheath 1010 into cavity or lumen 1020 (FIGS. 3B-4B). Port 1040 is configured to provide a throughway for a portion of trocar or trocar sleeve 100, or any alternative surgical instrument such as, for example, forceps, staplers, graspers, etc., disposed within cavity or lumen 1020 of surgical sleeve 1000 to be positioned outside cavity or lumen 1020. Port 1040 may be further configured to provide a sterile throughway, such that a sterile barrier is maintained. It should be appreciated that port 1040 permits a portion of trocar or trocar sleeve 100, or an alternative surgical instrument, to be exposed from cavity or lumen 1020, while maintaining the integrity of sheath 1010 with respect to absorbing or dampening impact thereon, or the ability for surgical sleeve 1000 to maintain a sterile barrier.

In an embodiment, surgical sleeve 1000 further includes a contact sensor 1050 (FIGS. 3B and 3C), wherein the contact sensor 1050 is disposed on at least one of sheath 1010 or contact surface 1200. Contact sensor 1050 is configured to measure a magnitude of impact force imparted upon surgical sleeve 1000, and/or detect the presence of imminent or actual physical contact thereon. Contact sensor 1050 may include, for example, a strain gauge, a piezocapactive pressure sensor, a force gauge or force sensor, a proximity sensor, a motion detector, a passive infrared sensor, a reed switch, a capacitance sensor, an electro-optical sensor, combinations thereof, or the like. Contact sensor 1050 may be configured to communicate with robotic surgical system 1 to alert the clinician of imminent or actual interaction of the sleeve with another object. In embodiments, robotic surgical system 1 includes a controller to inhibit, restrict or completely disable movement of the surgical instrument upon receipt of a signal from the contact sensor. In other embodiments, contact sensor 1050 includes visual indicia or auditory components thereon to alert the clinician of imminent or actual interaction of the sleeve with another object. It should be understood that multiple contact sensors can be employed on a single contact surface, or where multiple contact surfaces are present on the sleeve, individual contact sensors can be present on each of contact surfaces.

With reference to FIGS. 1-4B, a method for inhibiting, absorbing, or dampening impact force will be disclosed. With surgical sleeve 1000 in the un-deployed configuration, a surgeon, nurse, or other user (“operator”) initially positions sheath 1010 proximate to at least one of trocar or trocar sleeve 100, trocar mount 50, robotic arm 2, or any other component or assembly of robotic surgical system 1. Next, the operator transitions surgical sleeve 1000 into the deployed configuration such that at least a portion of trocar or trocar sleeve 100 is disposed within cavity or lumen 1020 of sheath 1010. The operator may unfurl sheath 1010 from the un-deployed configuration defining length “L₁” such that the length of cavity or lumen 1020 enlarges or extends to a desired second, larger length “L₂”, thus enclosing or enshrouding a portion of trocar or trocar sleeve 100. Alternatively or additionally, the operator may wrap first radial end portion 1015 of sheath 1010 about a portion of trocar or trocar sleeve 100 and affix first radial end portion 1015 and second radial end portion 1017 of sheath 1010 such that cavity or lumen 1020 of sheath 1010 is formed around at least a portion of trocar or trocar sleeve 100. Further, the operator may affix surgical sleeve 1000 to trocar or trocar sleeve 100 such that longitudinal translation of surgical sleeve 1000 with respect to trocar or trocar sleeve 100 is inhibited, via a radially inward bias of sheath 1010 or attachment element 1030. With at least a portion of trocar or trocar sleeve 100 disposed within cavity or lumen 1020 of surgical sleeve 1000, contact surface 1200 acts to inhibit, absorb, or dampen any impact force thereon resulting from inadvertent contact between trocar or trocar sleeve 100 and the patient, the operator, or any other surgical instrument or component of robotic surgical system 1. Further, contact sensor 1050 may be utilized to measure a magnitude of force thereon or detect the presence of physical contact therewith, and may further provide visual or auditory indicia to the operator. Further still, with surgical sleeve 1000 in the deployed configuration and positioned over at least a portion of trocar or trocar sleeve 100, a sterile barrier between trocar or trocar sleeve 100 and the operating theater may be achieved and maintained. After the completion of the procedure, surgical sleeve 1000 may be discarded or sterilized for reuse.

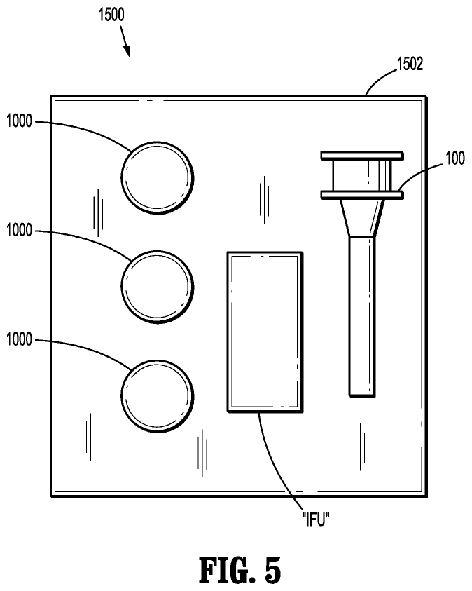

With reference to FIG. 5, a kit 1500 for absorbing and dampening impact force will be disclosed. Kit 1500 includes at least one surgical sleeve 1000, where surgical sleeve 1000 may define a range of longitudinal and radial dimensions to provide the operator with a variety of sizes to tailor surgical sleeve 1000 for a given surgical instrument. Kit 1500 may be configured such that surgical sleeve 1000 is provided in the un-deployed configuration, such that a low profile and compact packaging can be utilized. Further still, kit 1500 may include a sterile packaging 1502 configured to retain at least one surgical sleeve 1000, such that surgical sleeve 1000 is provided to the operator in a sterile fashion. Further, kit 1500 may include a plurality of accessories “A”, such as, trocars, trocar sleeves, surgical instruments and components (e.g., forceps or staplers), robotic components, or combinations thereof. In embodiments, kit 1500 may include an instruction for use (“IFU”) of surgical sleeve 1000, which may be packaged separately from, or together with surgical sleeve 1000. The IFU may include instructions directing an operator how to install surgical sleeve 1000, how to transition surgical sleeve 1000 from the un-deployed configuration to the deployed configuration, how to remove surgical sleeve 1000, how to sterilize surgical sleeve 1000, and the like.

Persons skilled in the art will understand that the structures and methods specifically described herein and shown in the accompanying figures are non-limiting exemplary embodiments, and that the description, disclosure, and figures should be construed merely as exemplary of particular embodiments. It is to be understood, therefore, that the present disclosure is not limited to the precise embodiments described, and that various other changes and modifications may be effected by one skilled in the art without departing from the scope or spirit of the disclosure. Additionally, the elements and features shown or described in connection with certain embodiments may be combined with the elements and features of certain other embodiments without departing from the scope of the present disclosure, and that such modifications and variations are also included within the scope of the present disclosure. Accordingly, the subject matter of the present disclosure is not limited by what has been particularly shown and described.

What is claimed is:

1. A surgical sleeve, comprising:
a sheath defining a lumen therein, the lumen configured to receive at least a portion of a surgical instrument therein, wherein the sheath is expandable to transition from an un-deployed configuration to a deployed configuration such that a length of the lumen increases as the sheath transitions from the un-deployed configuration to the deployed configuration; and
a contact surface disposed along at least a portion of the sheath and configured to dampen an impact force thereon.

2. The surgical sleeve of claim 1, wherein the sheath is furled onto itself in the un-deployed configuration and unfurls as the sheath transitions towards the deployed configuration.

3. The surgical sleeve of claim 1, wherein the contact surface includes a resilient foam element.

4. The surgical sleeve of claim 1, wherein the contact surface includes a rubber element.

5. The surgical sleeve of claim 1, wherein the sheath includes a material configured to withstand sterilization.

6. The surgical sleeve of claim 1, wherein the sheath includes a material configured to maintain the lumen of the sheath in a sterile condition.

7. The surgical sleeve of claim 1, wherein the sheath defines at least one port therethrough, the port configured to receive a portion of the surgical instrument therethrough.

8. The surgical sleeve of claim 1, wherein the sheath includes a contact sensor thereon, the contact sensor configured to measure a magnitude of force or detect physical contact upon the sheath.

9. The surgical sleeve of claim 8, wherein the contact sensor is supported on the contact surface.

10. A surgical wrap, comprising:
a sheath including a longitudinal seam and defining a first radial end portion and a second radial end portion, the sheath transitionable between an un-deployed configuration and a deployed configuration, in the deployed configuration the first radial end portion of the sheath affixes to the second radial end portion of the sheath such that in the deployed configuration the sheath defines a lumen therethrough configured to enshroud at least a portion of a surgical instrument, and
a contact surface disposed along at least a portion of the sheath and configured to dampen an impact force thereon.

11. The surgical warp of claim 10, wherein the contact surface includes a resilient foam element.

12. The surgical warp of claim 10, wherein the contact surface includes a rubber element.

13. The surgical warp of claim 10, wherein the sheath includes a material configured to withstand sterilization.

14. The surgical warp of claim 10, wherein the sheath includes a contact sensor thereon, the contact sensor configured to measure a magnitude of force or detect the presence of physical contact upon the sheath.

15. The surgical warp of claim 14, wherein the contact sensor is supported on the contact surface.

16. The surgical warp of claim 10, wherein at least one of the first or second radial end portions of the sheath further includes an affixation element, the affixation element configured to selectively affix the first radial end portion to the second radial end portion in the deployed configuration.

17. The surgical warp of claim 16, wherein the affixation element includes a string.

18. The surgical warp of claim 16, wherein the affixation element includes an adhesive.

19. The surgical warp of claim 10, wherein the lumen of the sheath defines a shape complementary to the surgical instrument.

20. The surgical wrap of claim 10, wherein the sheath includes a material configured to maintain the lumen of the sheath in a sterile condition.

21. The surgical wrap of claim 10, wherein the sheath defines at least one port therethrough, the port configured to receive a portion of a surgical instrument therethrough.

22. A kit, comprising:
at least one surgical sleeve, the surgical sleeve including:
a sheath defining a lumen therein, the lumen configured to receive at least a portion of a surgical instrument therein, wherein the sheath is expandable to transition from an un-deployed configuration to a deployed configuration such that a length of the lumen increases as the sheath transitions from the un-deployed configuration to the deployed configuration; and
a contact surface disposed along at least a portion of the sheath and configured to dampen an impact force thereon, and
an instruction for directing a user to position the at least one surgical sleeve over a surgical instrument or transition the at least one surgical sleeve from the un-deployed configuration to the deployed configuration.

23. The kit of claim 22, further comprising:
at least one surgical instrument including a trocar or a trocar sleeve.

24. The kit of claim 22, further comprising:
a sterile packaging configured to retain the at least one surgical sleeve and the instruction for use.
